(19) United States
(12) Patent Application Publication
(10) Pub. No.: US 2022/0079880 A1
(43) Pub. Date: Mar. 17, 2022

(54) SOLID MICELLAR COMPOSITIONS OF CANNABINOID ACIDS

(71) Applicants: Thomas PICCARIELLO, Blacksburg, VA (US); Michaela MULHARE, Radford, VA (US); Scott PALMER, Blacksburg, VA (US); Synthonics, Inc., Blacksburg, VA (US)

(72) Inventors: Thomas PICCARIELLO, Blacksburg, VA (US); Michaela MULHARE, Radford, VA (US); Scott PALMER, Blacksburg, VA (US)

(21) Appl. No.: 17/423,846

(22) PCT Filed: Jan. 17, 2020
(86) PCT No.: PCT/US2020/014109

§ 371 Date: Jul. 16, 2021

Related U.S. Application Data
(60) Provisional application No. 62/793,460, filed on Jan. 17, 2019.

(51) Int. Cl. A61K 9/107; A61K 31/352; A61K 31/07; A61K 31/122; A61K 31/51; A61K 31/465; A61K 33/06; A61P 25/28; A61P 25/16; A61P 25/02

(52) U.S. Cl. CPC A61K 9/107; A61K 31/352; A61K 31/07; A61K 31/122; A61P 25/02; A61K 31/465; A61K 33/06; A61P 25/28; A61P 25/16; A61K 31/51

(57) ABSTRACT

Solid, micellar compositions, comprising micelles of one or more cannabinoid acids and a metal, wherein the cannabinoid acids are in a salt form, the salt form has a monovalent counter ion, and the micelles are free of added surfactants, are disclosed. Additionally, processes for preparing solid, micellar compositions, comprising micelles of one or more cannabinoid acids and a metal, wherein the cannabinoid acids are in a salt form, the salt form has monovalent counter ion, and the micelles are free of added surfactants, are disclosed. Finally, methods of treating a number of disorders comprising the step of administering to a patient in need thereof a therapeutically effective amount of a solid, micellar composition comprising micelles of one or more cannabinoid acids and a metal, wherein the cannabinoid acids are in a salt form, the salt form has monovalent counter ion, and the micelles are free of added surfactants, are disclosed.

### CROSS-REFERENCE TO RELATED APPLICATIONS

This application claims the benefit of U.S. Provisional Application No. 62/793,460, filed Jan. 17, 2019, which is incorporated by reference in its entirety.

### TECHNICAL FIELD

This disclosure relates to solid micellar compositions of cannabinoid acids, processes for preparing solid micellar compositions of cannabinoid acids, and methods of treating certain diseases with solid micellar compositions of cannabinoid acids.

### BACKGROUND

The medical benefits of cannabinoids have been known and practiced for centuries. More than 26,000 studies and reviews referencing cannabis plants and cannabinoid molecules have been published in the recent past, including over 6,100 articles in 2018. Indeed, this renewed interest in cannabinoid therapeutics can be principally attributed to the recent discovery of the endocannabinoid receptors throughout the central nervous system, combined with the growing number of observational reports from patients and their physicians as to the medicinal benefits of cannabis. Recent changes in the legal status of hemp and marijuana have encouraged scientists to conduct a growing number of clinical studies with the plant, its extracts and its isolates.

Additionally, the number of disease states for which clinical benefits have been described, both pre-clinical and clinical, has been growing and now includes Alzheimer's Disease, Amyotrophic Lateral Sclerosis (ALS), chronic pain, diabetes mellitus, dystonias and other movement disorders such as Parkinson's disease, treatment-resistant epilepsies, fibromyalgia, inflammatory bowel diseases, gliomas and other types of cancers, Hepatitis C infections, Human Immunodeficiency Virus (HIV) infections, Huntington's disease, hypertension, urinary incontinence, methicillin-resistant Staphylococcus aureus (MRSA) infections, migraine headaches, multiple sclerosis, osteoporosis, Post-Traumatic Stress Disorders (PTSD), chronic pruritus, rheumatoid arthritis, sleep apnea, autistic spectrum disorders, and Chronic Traumatic Encephalopathy (CTE).

Many of the clinical benefits attributed to cannabinoids stem from the pharmacological effects of cannabidiol (CBD), the decarboxylated congener of cannabidiolic acid (CBDa). While its mechanisms of action remain under study, it is proposed that CBD interacts with multiple central nervous system receptors, ion channels and neurotransmitters. Additionally, its effects on adenosine reuptake, and the GPR-55 and TRPV1 receptors are thought to contribute to its antiepileptic mechanisms. When tested at the National Institute of Neurological Disorders and Stroke (NINDS) drug testing laboratory, CBD showed an ED₅₀ for the MBS model of epilepsy of 85 mg/kg in mice and 89 mg/kg in rats. CBD was also active in the Metrazol model, the cardiogenic model, and other chemo-convulsant models at doses between 80 mg/kg and 200 mg/kg. These are the same positive metrics of testing that have been identified in almost all current anticonvulsants.

Most current surveys and studies show a positive effect of CBD on the frequency and severity of seizures, although the data has been limited by the lack of clarity as to the exact dose of CBD necessary to affect therapeutic clinical responses. Almost all surveys, however, objectively show an approximate 47% response rate with a reduced seizure frequency of greater than 50%. This is a robust and significant response when compared to other anti-seizure drug testing. A study from Israel using an enriched CBD/tetrahydrocannabinol (THC) preparation in 74 patients with epileptic encephalopathies reported a decrease in seizure frequency of approximately 50% in 50% of treated patients, with concomitant improvement in behavior, alertness, communication, language, motor skills, and sleep.

As touched upon, recent identification of an integrated endocannabinoid regulatory system has helped drive recognition and understanding of many of the remarkable medical benefits of cannabinoids. This is explained, in part, by the cannabinoid molecules' variable affinity for cannabinoid receptors expressed throughout the human body (although, as recently discovered, cannabinoid receptors are even expressed in very rudimentary species of animals). More recently, it has been discovered that other receptors, such as PPARγ, 5HT_{1A}, GPCR, and COX-2, are likewise fundamental to cannabinoids' diverse medical effects and mechanisms of action. Moreover, all cannabinoid acids, namely cannabidolic acid (CBDa), tetrahydrocannabinolic acid (THCa) and cannabigerolic acid (CBGa), outperform their corresponding neutral cannabinoids in terms of PPARy binding. This is particularly true for THCa, which has shown potent neuroprotective activity, possibly through down regulation of the expression of inflammatory genes. Thus, THCa has significant potential to treat progressive neurodegenerative disorders such as Huntington's disease and other neuroinflammatory conditions. Additionally, THCa has been shown in animal studies to limit the spread of prostate cancer cells.

CBDa, the predominant cannabinoid in many cannabis strains, demonstrates the importance of the interplay between cannabinoids and cannabinoid receptors. Much of the work demonstrating CBDa's analgesic effect was conducted by Linda Parker and her associates who noted that THC's anti-hyperalgesia was blocked by CB1 receptor antagonists, whereas CBDa's analogous effects were blocked by TRPV1 antagonists (Rock et al., Psychopharmacology, 2018, 235, 3259-3271). This apparent selectivity of CBDa at these transfer receptor sites is supported by Izzo, wherein it was shown that CBDa was an agonist for TRPA1 (nociception attenuation), TRPV1′ (cancer cell apoptosis inducer) and TRPM8 (Izzo, Cell Press, 2009, 515-527). CBDa, for which specific and novel anti-inflammatory, anti-epileptic, anti-emetic, analgesic, anti-proliferative and anti-microbial effects have now been described, may hold a unique role in treating acute pain, psychosis and various carcinomas apart from the other cannabinoids.

Bolognini reported that CBDa suppressed experimentally induced nausea in rats in a 5HT_{1A} receptor-mediated manner (Bolognini et al., British Journal of Pharmacology, 2013, 168(6), 1456-1470). It was further reported that compared with CBD, CBDa may display greater potency, efficacy and selectivity at ameliorating signs of cerebral infarction, anxiety and depression via 5HT_{1A} receptor-dependent mechanisms in animal models.

Considerable research on CBDa's interaction with COX-2 receptors has been conducted by Kazuhito Watanabe and associates at Hokuriku University. Specifically, in 2008 these researchers reported that CBDa (˜2 micromolar concentration) inhibited COX-2. It was further noted that full inhibition of the COX-2 receptor required the carboxylic acid moiety, thus CBD is not nearly as active (Takeda et al., Drug Metabolism and Disposition, 2008, 36(9), 1917-1921). Moreover, CBDa is selective for COX-2 and does not inhibit COX-1, which is beneficial since inhibition of COX-1 may lead to unwanted side effects such as GI ulceration/bleeding and platelet dysfunction. Thus, the advantages of CBDa's selective inhibition of COX-2 may be quite substantial. CBDa's dual inhibitory effects of COX-2 may also suppress genes associated with cancer metastasis. It was further determined that this COX-2 inhibition involves the down regulation of specific proto-oncogenes that promote the progression of some cancers. Recently it has also been shown that CBDa has the unique ability to inhibit migration of highly invasive MDA-MB-231 human breast cancer cells.

Although there are scant reports on bioavailability on many of the cannabinoids, the relative bioavailability of CBD has been reported to be only between 8 and 10%. Even though CBDa may be metabolized by a different set of liver enzymes than CBD, it would be reasonable to expect that CBDa would not exhibit a dramatic improvement in bioavailability. Indeed, Nahler reported that the relative oral bioavailability of CBDa to be 19% (Nahler, BioBloom Hemp, 2017, 1-6).

Successful efforts to improve the bioavailability of CBD and cannabinoids have included co-administration with lipids, infusing CBD or THC oils with edible oils and fats, formulating CBD into liposomes or micelles, and incubating these cannabinoid molecules with chylomicron isolates. For example, it has been shown that dietary fats and pharmaceutical lipid excipients increase gastrointestinal absorption of orally administered cannabinoids. Pursuing this line of thinking, PoViva Tea infuses cannabinoids with bioavailability enhancing reagents such as non-fat dry milk, triglycerides and natural oils (Reillo et al., U.S. Pat. No. 9,4747,725).

Liposomal drug delivery (LDD) systems are widely used for enhancing delivery of lipophilic drugs into the bloodstream or similarly delivered hydrophilic drugs through encapsulation of the drug into the aqueous layer of the liposome. Liposomes incorporate lipid molecules into their hydrophobic core; the lipid molecules are, in effect, dissolved in the hydrophobic core of the liposome. Whereas, LDD systems are an effective hydrophobic drug delivery method, they are somewhat limited by drug loading capacity, stability and in vivo behavior. LDD systems simulate the manner in which cholesterol and fatty acids are incorporated into chylomicrons. Nascent chylomicrons are micellar structures formed naturally in the small intestines though a bile acid-controlled emulsification of cholesterol, apolipoproteins and triglycerides (FIG. 1). Nascent chylomicrons are processed into mature chylomicrons after absorption into the intestinal epithelial cells, and then enter the bloodstream through the lymphatic system. Chylomicrons and their contents, thereby, effectively bypass hepatic circulation as they enter the bloodstream.

Several approaches to forming liposome formulations of CBD oil are currently commercially available. In general, these technologies utilize the standard methods of forming liposomes, which are synthetic analogs of micelles, wherein a surfactant (e.g phospholipid) and a lipid (e.g CBD) are mixed with water under proper equilibrating conditions, thereby assembling the complex into a spherical structure with a hydrophilic core and exterior separated by a lipophilic shell. This is where the lipophilic cannabinoid (i.e. CBD) resides. These liposome formulations allow for greater water solubility of the CBD molecule and, in some cases, are designed to emulate the lymphatic transport mode of chylomicrons. Submicron sized liposomes of CBD are created by nano-emulsification of CBD using phosphatidylcholine as the surfactant. Recently, nano-size lipid-based systems have been developed to improve the bioavailability (BA) of cannabinoids (e.g. CBD had a 10× improvement in BA).

Alternatively, one can improve the solubility and, consequently, the bioavailability of CBDa, by incorporating it into the hydrophobic core of micelles. This approach is exemplified by the VESlsorb technology (Source One Global Partners), which is designed to mimic naturally occurring chylomicrons in the human body. The VESlsorb delivery system was introduced with the demonstration of improved bioavailability of CoQ10 over competing solubilizing methods. Enschara and other companies incorporate the VesiSorb delivery system into their CBD delivery products producing colloidal-CBD droplets. Ojai Energetics (OE) also commercializes a colloidal or micellar CBD product. Thus, both OE and VesiSorb technologies reportedly closely copy the process of nascent chylomicrons. Since chylomicrons are transported into the bloodstream via the lymphatic system, first-pass metabolism by the liver can be avoided. Molecules within the chylomicrons absorbed by the lymphatic system typically have improved bioavailability (purportedly up to 20-fold) over molecules that undergo hepatic circulation for this reason.

Strauss discovered in 1977 that everted hamster jejunum incubated with fatty acids and monoglycerides in bile acid solutions produced very few chylomicrons at low concentrations of calcium and magnesium, but released numerous chylomicron-like particles when the concentration of the ions were at physiologic levels (Strauss, Gastroenterology, 1977, 73(2), 421-424). Given the chelating capacity of magnesium and calcium, it seems reasonable that these metals facilitate aggregation of lipids in the small intestines into micelles which are suitable for the formation of chylomicrons. This phenomenon is further supported by the selective precipitation of chylomicrons and very low-density lipoprotein (VLDL) by magnesium ions.

### SUMMARY

Solid, micellar compositions, comprising micelles of one or more cannabinoid acids and a metal, wherein the cannabinoid acids are in a salt form, the salt form has a monovalent counter ion, and the micelles are free of added surfactants, are disclosed herein. In some embodiments, the compositions further comprise additional lipid components selected from cannabinoids, terpenes, vitamins, fatty acids, nicotine, resveratrol, tocotrienols, flavonoids, gamma-tocopherols, steroids, lipophilic antibiotics, and pharmaceutical compounds.

Additionally, processes for preparing solid, micellar compositions, comprising micelles of one or more cannabinoid acids and a metal, wherein the cannabinoid acids are in a salt form, the salt form has monovalent counter ion, and the micelles are free of added surfactants, are disclosed herein. The processes include the steps of adding the cannabinoid acids to a solution comprising water, converting the cannabinoid acids to a salt form, preferably by the addition of a base with a monovalent cation, emulsifying the salt form of the cannabinoid acids to form micelles, filtering the micelles, adding a metal to the micelles to form a precipitate, and isolating the precipitate from the solution. In some embodiments, the processes include the additional step of adding one or more additional lipid components to the solution, wherein the additional lipid components are selected from the group consisting of cannabinoids, terpenes, vitamins, fatty acids, nicotine, resveratrol, tocotrienols, flavonoids, gamma-tocopherols, steroids, lipophilic antibiotics, and pharmaceutical compounds.

Finally, methods of treating a number of neurodegenerative disorders comprising the step of administering to a patient in need thereof a therapeutically effective amount of a solid, micellar composition comprising micelles of one or more cannabinoid acids and a metal, wherein the cannabinoid acids are in a salt form, the salt form has monovalent counter ion, and the micelles are free of added surfactants, are disclosed herein. In some embodiments, the neurodegenerative disorders to be treated are selected from the group consisting of amyotrophic lateral sclerosis, Parkinson's disease, multiple sclerosis, and stroke. In some embodiments, the methods of treatment disclosed herein involve administering solid, micellar compositions that further comprise additional lipid components selected from cannabinoids, terpenes, vitamins, fatty acids, nicotine, resveratrol, tocotrienols, flavonoids, gamma-tocopherols, steroids, lipophilic antibiotics, and pharmaceutical compounds.

### BRIEF DESCRIPTION OF THE DRAWINGS

FIG. 1 shows the structure of a chylomicron, a spherical structure composed of lipoproetins, fats, cholesterol, and triglycerides produced naturally within the body.

FIG. 2 shows a schematic representation of a chylobinoid micelle composed of sodium cannabidolic acid. The polar carboxylate forms the outer shell of the micelle and is solvated by water. The lipophilic portion of the CBDa molecule forms the inner core of the micelle.

FIG. 3 shows a schematic representation of a chylobinoid micelle, wherein the lipophilic core has solubilized a lipophilic adjuvant such as other cannabinoids, curcumin, and vitamins.

FIG. 4 shows a schematic representation of a metal-complexed chylobinoid micelle, wherein the metal forms an outer shell surrounding the chylobinoid micelle. The multiple bonding modes, namely the formation of intermolecular bonds between CBDa molecules of the micelle (left) and intramolecular bonding (right), contribute to the solidification and stabilization of the micelle structure.

FIG. 5 shows a process diagram for the preparation of chylobinoid micelles.

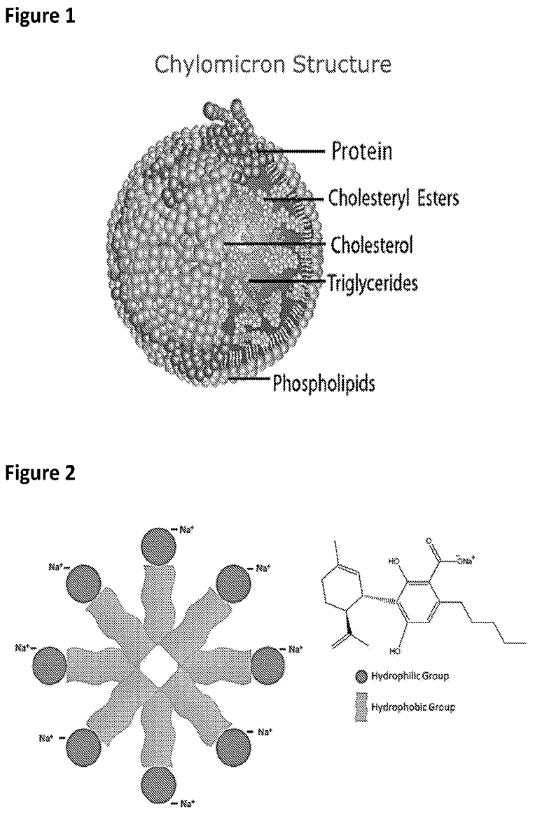

### DETAILED DESCRIPTION

The present disclosure relates to the discovery that ionized CBDa molecules derived from emulsified hemp plant extracts spontaneously form micelle-like compounds similar to the precursors of chylomicrons. The surfactant used in the process of forming the micelle is the salt of CBDa. Through the application of principles established for the formation of stable, metal-coordinated cannabinoid pharmaceuticals, we have discovered that CBDa's chemical structure is ideally suited for forming metal coordinated congeners. Accordingly, upon the addition of magnesium ions to an emulsion of micellar, ionized CBDa, a precipitate of magnesium coordinated CBDa-rich micelles forms.

These micellar, metal-coordinated CBDa constructs have inherent properties akin to chylomicrons and nascent chylomicrons, such that upon oral delivery and absorption through the intestinal epithelia they will be transported into the bloodstream via the lymphatic system, thereby bypassing first-pass metabolism in the liver.

### Compositions of the Disclosure

The present disclosure relates to the discovery of two phenomena that greatly improve the efficiency of cannabinoid micelle formation. The first is that the addition of a mild base to a mixture of crude marijuana or hemp extract in water creates an emulsion of cannabinoid acid-based micelles. Without being bound to theory, the mild base and the acidic components in the crude marijuana or hemp extract, in effect, mimic the physiological role that bile acids and/or phospholipids play in the intestines for the formation of chylomicrons. In other words, structures resembling true micelles are being formed from marijuana or hemp extracts by replacing the phospholipids and salts of bile acid with the salt of CBDa (if from hemp) or THCa (if from marijuana), as shown in FIG. 2. This is supported by a study showing that ⅓ of cannabinoids co-administered with dietary fats or pharmaceutical lipid excipients are distributed in the micellar component (Zgair et al., Am. J. Transl. Res., 2016, 8(8), 3448-3459). As discussed above, lipid formulations significantly increase the bioavailability of cannabinoids.

The second phenomenon observed is that adding any of the S2 block metal halides (e.g. magnesium ions, calcium ions, strontium ions) to the emulsion of cannabinoid acid-based micelles creates a precipitant. Thus, we have created cannabinoid-containing chylomicrons, named “chylobinoids.”

Moreover, metal coordinated cannabinoid micelles, that is chylobinoids, increase the stability of the lipid complex, which in turn increases the bioavailability of cannabinoids by retaining their content above the observed ⅓ in the micelle after lipolysis. Without being bound to theory, the precipitation of the CBDa-micelles formed in the emulsification process produces solid-state micelles, with the structure and contents thereof preserved through magnesium coordination of the carboxylate and hydroxy moieties of the CBDa molecule. The solid-state micelles have improved stability, and thereby have longer shelf lives, greater loading capacity, and improved in vivo performance, vis-a-vis the current LDD or micellar (e.g. VESIsorb) technologies. Further improvement to the stability of the solid, metal-coordinated, CBDa-containing micelle is provided by intermolecular bonding between the metal and neighboring CBDa molecules.

Furthermore, chylobinoids are absorbed similarly to the cannabinoids formulated into nano-size lipid-based systems discussed above. The difference is that the chylobinoid compositions described herein require no addition of organic emulsifying reagents, are less complicated to practice, and are adaptable to almost any scale.

Solid, micellar compositions, comprising micelles of one or more cannabinoid acids and a metal, wherein the cannabinoid acids are in a salt form with a monovalent counter ion, and the micelles are free of added surfactants, are disclosed herein.

In some embodiments, the solid, micellar compositions comprise micelles of one or more cannabinoid acids selected from the group consisting of cannabidolic acid (CBDa), tetrahydrocannabinolic acid (THCa) and cannabigerolic acid (CBGa). In some embodiments, the cannabinoid acid is CBDa. In some embodiments, the cannabinoid acid is THCa. In some embodiments, the cannabinoid acid is CBGa. In some embodiments the solid micellar compositions comprise micelles of a combination of CBDa, THCa, and/or CBGa.

In some embodiments, the monovalent counter ion of the salt form of the cannabinoid acid is selected from the group consisting of lithium, sodium, potassium, rubidium, cesium, and ammonium. In some embodiments, the monovalent counter ion is lithium. In some embodiments, the monovalent counter ion is sodium. In some embodiments, the monovalent counter ion is potassium. In some embodiments, the monovalent counter ion is rubidium. In some embodiments, the monovalent counter ion is cesium. In some embodiments, the monovalent counter ion is ammonium.

In some embodiments, the solid, micellar compositions comprise micelles of one or more cannabinoid acids and a metal, wherein the metal is selected from an s-block metal, a d-block metal, and a p-block metal. In some embodiments, the metal is an s-block metal. In some embodiments, the metal is a d-block metal. In some embodiments, the metal is a p-block metal.

In some embodiments, the solid, micellar compositions comprise micelles of one or more cannabinoid acids and a metal, where the metal is selected from the group consisting of magnesium, calcium, and strontium. In some embodiments, the metal is magnesium. In some embodiments, the metal is calcium. In some embodiments, the metal is strontium.

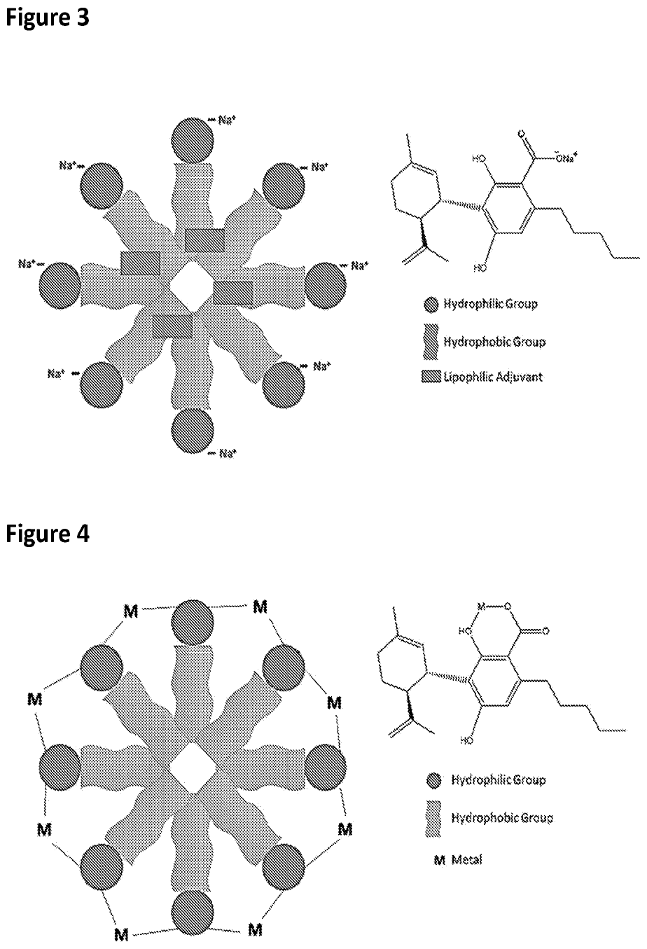

Given the nature of emulsified CBDa salt hemp extract, additional lipophilic components are solubilized by the non-polar moiety of the CBDa molecule and sequestered in the inner core of the micelle (as shown in FIG. 3). Upon precipitation of the CBDa micelle with magnesium, additional cannabinoids, terpenes, and other naturally occurring lipids and waxes, are trapped within the solid CBDa micelle. The incorporation of the other cannabinoids and lipids into the chylobinoid composition can contribute to the “entourage effect”, thereby enhancing the composition's pharmacological activity. The compositions and methods described herein allow for the retention of medically beneficial components from the hemp or marijuana plants, with concomitant removal of extraneous plant waste material. Chylobinoids are therefore a highly purified construct of the pharmacologically active components in crude hemp and marijuana extracts.

Given that additional cannabinoids, terpenes, and other lipids are retained within the CBDa salt micelle, by virtue of precipitation of the chylobinoid complex with magnesium, other lipophilic compounds can also be included within the chylobinoid. As described in Example 3 below, curcumin was added to a hemp emulsion and became soluble, indicating that it was incorporated into the CBDa salt micelle (FIG. 3). Moreover, the curcumin was retained in the chylobinoid complex upon precipitation via the addition of magnesium. Based upon the observed decrease in CBDa content following the addition of curcumin (˜70% to ˜30% reduction), an approximate curcurmin:CBDa ratio of 2:1 was produced in the isolated curcumin infused chylobinoid powder.

In some embodiments, the solid, micellar compositions comprising micelles of one or more cannabinoid acids and a metal described herein further comprise one or more additional lipid components, wherein the additional lipid components are sequestered within the hydrophobic core of the micelles.

In some embodiments, the additional lipid components are cannabinoids and terpenes. In some embodiments, the additional lipid components are cannabinoids. In some embodiments, the additional lipid components are cannabinoids selected from the group consisting of THC, CBD, THCa, CBGa, CBG, CBN. In some embodiments, the additional lipid components are terpenes. In some embodiments, the additional lipid components are terpenes selected from the group consisting of α-pinene, myrcene, carophyllene oxide, limonene, linalool, β-carophyllene, α-humulene and terpinolene. In some embodiments, the additional lipid components are a mixture of cannabinoids and terpenes. In some embodiments, the additional lipid components are a mixture of cannabinoids and terpenes selected from the group consisting of THC, CBD, THCa, CBGa, CBG, CBN, α-pinene, myrcene, carophyllene oxide, limonene, linalool, β-carophyllene, α-humulene and terpinolene. In some embodiments, the additional lipid components are cannabinoids and/or terpenes, wherein the cannabinoids and terpenes are obtained from a plant concurrently with the cannabinoid acids that form the micelles. In some embodiments, the additional lipid components are cannabinoids and/or terpenes, wherein the cannabinoids and terpenes are added to the micelles.

In some embodiments, the additional lipid components are selected from the group consisting of CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, vitamin K. In some embodiments, the additional lipid components are selected from the group consisting of CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, vitamin K, and others.

In some embodiments, lipophilic pharmaceuticals can be sequestered within the CBDa salt micelle and the resulting solid chylobinoid complex. In some embodiments, the biological activity of the lipophilic additives act synergistically with the cannabinoids contained in the chylobinoid complex, by virtue of the concurrent or sequential delivery of the components contained within the chylobinoid complex. In some embodiments, the other cannabinoids, including CBD, act synergistically with each other and the chylobinoids by virtue of the concurrent or sequential delivery of the components contained within the chylobinoid complex. In some embodiments, the metal (e.g. magnesium or other s-, s2-, d-, or p-block metal) ion itself potentiates the effects of the cannabinoid molecule and the lipophilic compound(s) contained within the chylobinoid. In some embodiments the metal (e.g. magnesium or other s-, s2-, d, or p-block metal) may independently exert its own biological effects in combination or in concert with the cannabinoid and lipophilic molecule(s) contained within the chylobinoid.

All manner of biologically active agents are contemplated for use in accordance with the present teachings—preferably ones that have inadequate solubilities at physiological pH and which could potentially benefit from incorporation into chylobinoid in accordance with the present teachings. Representative agents contemplated for use include but are not limited to the following: medicaments for treating the gastrointestinal (GI) tract (e.g., antacids; reflux suppressants; antiflatulents; antidopaminergics; proton pump inhibitors (PPIs); H2-receptor antagonists; cytoprotectants; prostaglandin analogues; laxatives; antispasmodics; antidiarrheals; bile acid sequestrants; opioids; and the like); medicaments for treating the cardiovascular system (e.g., β-receptor blockers; calcium channel blockers; diuretics; cardiac glycosides; antiarrhythmics; nitrate; antianginals; vasoconstrictors; vasodilators; peripheral activators; and the like); antihypertension agents (e.g., ACE inhibitors; angiotensin receptor blockers; a blockers; and the like); coagulation agents (e.g., anticoagulants; heparin; antiplatelet drugs; fibrinolytics; anti-hemophilic factors; haemostatic drugs; and the like); atherosclerosis/cholesterol inhibitors (e.g., hypolipidaemic agents; statins; and the like); medicaments that affect the central nervous system (e.g., hypnotics; anesthetics; antipsychotics; antidepressants including but not limited to tricyclic antidepressants, monoamine oxidase inhibitors, selective serotonin reuptake inhibitors, etc.; and the like); antiemetics; anticonvulsants; antiepileptics; anxiolytics; barbiturates; movement disorder drugs including but not limited to those for treating Parkinson's disease, etc.; stimulants including but not limited to amphetamines; benzodiazepines; cyclopyrrolones; dopamine antagonists; antihistamines; cholinergics; anticholinergics; emetics; cannabinoids; 5-HT serotonin antagonists; and the like); analgesics (e.g., nonsteroidal antiinflammatory drugs or NSAIDs; opioids; various orphan drugs including but not limited to paracetamol, tricyclic antidepressants, anticonvulsants, etc.; and the like); medicaments for treating musculoskeletal disorders (e.g., NSAIDs including but not limited to COX-2 selective inhibitors, etc.; muscle relaxants; neuromuscular drugs; anticholinesterases; and the like); medicaments for treating the eye (e.g., adrenergic neurone blockers; astringents; ocular lubricants; mydriatics; cycloplegics; anti-glaucoma agents including but not limited to adrenergic agonists, beta-blockers, carbonic anhydrase inhibitors/hyperosmotics, cholinergics, miotics, parasympathomimetics, prostaglandin agonists/prostaglandin inhibitors, nitroglycerin, etc.; and the like); topical anesthetics (e.g., benzocaine; butamben; dibucaine; lidocaine; oxybuprocaine; pramoxine; proparacaine; proxymetacaine; tetracaine; and the like); sympathomimetics; parasympatholytics; anti-bacterial agents (e.g., antibiotics; topical antibiotics; sulfa drugs; aminoglycosides; fluoroquinolones; and the like); antiviral drugs; medicaments for treatment of the ear, nose, and throat (e.g., sympathomimetics; antihistamines; anticholinergics; NSAIDs; steroids; antiseptics; local anesthetics; antifungals; cerumenolyti; and the like); medicaments for treating the respiratory system (e.g., bronchodilators; NSAIDs; anti-allergics; antitussives; mucolytics; decongestants; corticosteroids; β-2-adrenergic agonists; anticholinergics; steroids; and the like); medicaments for treating diseases of the endocrine system (e.g., androgens; antiandrogens; gonadotropin; corticosteroids; human growth hormone; insulin; antidiabetics including but not limited to sulfonylureas, biguanides/metformin, thiazolidinediones, insulin, etc.; thyroid hormones; antithyroid drugs; calcitonin; diphosponate; vasopressin analogues; and the like); medicaments for treating the reproductive system and urinary system (e.g., antifungals; alkalizing agents; quinolones; antibiotics; cholinergics; anticholinergics; anticholinesterases; antispasmodics; 5-α reductase inhibitor; selective α-1 blockers; sildenafils; fertility medications; and the like); contraceptives (e.g., hormonal contraceptives; and the like); medicaments for use in obstetrics and gynecology (e.g., NSAIDs; anticholinergics; haemostatic drugs; antifibrinolytics; hormone replacement therapy (HRT); bone regulators; β-receptor agonists; follicle stimulating hormone; luteinizing hormone; luteinizing-hormone-releasing hormone (LHRH); gonadotropin release inhibitor; progestogen; dopamine agonists; oestrogen; prostaglandins; gonadorelin; diethylstilbestrol; and the like); medicaments for treating the skin (e.g., emollients; anti-pruritics; antifungals; disinfectants; scabicides; pediculicides; tar products; vitamin A derivatives; vitamin D analogues; keratolytics; abrasives; systemic antibiotics; topical antibiotics; hormones; desloughing agents; exudate absorbents; fibrinolytics; proteolytics; sunscreens; antiperspirants; corticosteroids; and the like); medicaments for treating infections and infestations (e.g., antibiotics; antifungals including but not limited to imidazoles, polyenes, etc.; antileprotics; antituberculous drugs; antimalarials; anthelmintics; amoebicides; antivirals; antiprotozoals; antiparasitics; and the like); anti-inflammatory agents (e.g., NSAIDs; corticosteroids; and the like); medicaments for treating the immune system (e.g., vaccines; immunoglobulins; immunosuppressants; interferons; monoclonal antibodies; and the like); medicaments for treating allergies (e.g., anti-allergics; antihistamines; NSAIDs; mast cell inhibitors; and the like); nutritional agents (e.g., tonics; iron preparations; electrolytes; parenteral nutritional supplements; vitamins; anti-obesity drugs; anabolic drugs; haematopoietic drugs; food product drugs; and the like); antineoplastic agents (e.g., cytotoxic drugs; therapeutic antibodies; sex hormones; aromatase inhibitors; somatostatin inhibitors; recombinant interleukins; G-CSF; erythropoietin; and the like); euthanaticum agents; and the like; and combinations thereof.

A property inherent in many metals is the capacity to bind multiple ligands. This is best exemplified by the enhanced solubility imparted to metal-coordinated pharmaceuticals by incorporation of water-soluble ligands into the metal-coordinated complex. This property of expanding the coordination sphere to enhance physicochemical properties is inherent in chylobinoids, as well. It is therefore an embodiment of this invention that the solubility of chylobinoid is enhanced through the incorporation of water-soluble ligands, bound to the magnesium (or other S2 block metal) atom, to the chylobinoid complex.

### Processes for Preparing the Compositions of the Disclosure

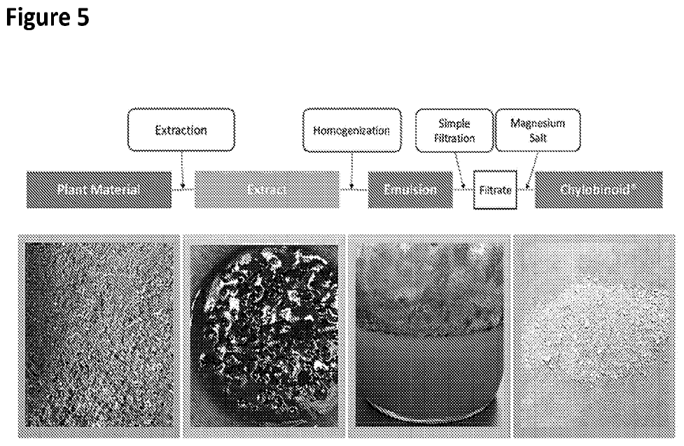

Micelles formed by processes using classic surfactants such as phospholipids are typically used in their application as is; that is, in a liquid formulation. A characteristic of this invention is that magnesium chelation is utilized to form coordination complexes between the magnesium atom and the carboxylate and hydroxyl moieties of the CBDa molecule, as depicted in FIG. 4. Further improvement to the stability of the solid, metal-coordinated, CBDa-containing micelle is provided by intermolecular bonding between the metal and neighboring CBDa molecules, as also depicted in FIG. 4. Thusly, upon addition of a magnesium salt to the emulsified CBDa salt, a white or off-white precipitant is formed. The product of this magnesium salt-induced precipitation is the solid-state magnesium coordinated micelle, which is defined as a chylobinoid. The other lipids, including other cannabinoids that were incorporated into the CBDa salt micelle are also incorporated into the chylobinoid. The process is summarized pictorially in FIG. 5.

Processes for preparing solid, micellar compositions comprising micelles of one or more cannabinoid acids and a metal, wherein the cannabinoid acids are in a salt form with a monovalent counter ion, and the micelles are free of added surfactants, are disclosed herein. The processes include the steps of:
adding one or more cannabinoid acids to a solution comprising water;
converting the one or more cannabinoid acids to a salt form;
emulsifying the salt form of the one or more cannabinoid acids to form the micelles; filtering the micelles;
adding the metal to the micelles to form a precipitate; and isolating the precipitate from the solution.

In some embodiments, the one or more cannabinoid acids are selected from the group consisting of cannabidolic acid (CBDa), tetrahydrocannabinolic acid (THCa) and cannabigerolic acid (CBGa). In some embodiments, the cannabinoid acid is CBDa. In some embodiments, the cannabinoid acid is THCa. In some embodiments, the cannabinoid acid is CBGa. In some embodiments the solid micellar compositions comprise micelles of a combination of CBDa, THCa, and/or CBGa.

In some embodiments, the solid, micellar compositions comprising micelles of one or more cannabinoid acids and a metal described herein further comprise one or more additional lipid components, wherein the additional lipid components are sequestered within the hydrophobic core of the micelles.

In some embodiments, the additional lipid components are cannabinoids and terpenes. In some embodiments, the additional lipid components are cannabinoids. In some embodiments, the additional lipid components are cannabinoids selected from the group consisting of THC, CBD, THCa, CBGa, CBG, CBN. In some embodiments, the additional lipid components are terpenes. In some embodiments, the additional lipid components are terpenes selected from the group consisting of α-pinene, myrcene, carophyllene oxide, limonene, linalool, β-carophyllene, α-humulene and terpinolene. In some embodiments, the additional lipid components are a mixture of cannabinoids and terpenes. In some embodiments, the additional lipid components are a mixture of cannabinoids and terpenes selected from the group consisting of THC, CBD, THCa, CBGa, CBG, CBN, α-pinene, myrcene, carophyllene oxide, limonene, linalool, β-carophyllene, α-humulene and terpinolene. In some embodiments, the additional lipid components are cannabinoids and/or terpenes, wherein the cannabinoids and terpenes are obtained from a plant concurrently with the cannabinoid acids that form the micelles. In some embodiments, the additional lipid components are cannabinoids and/or terpenes, wherein the cannabinoids and terpenes are added to the micelles.

In some embodiments, the additional lipid components are selected from the group consisting of CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, vitamin K. In some embodiments, the additional lipid components are selected from the group consisting of CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, vitamin K, and others.

In some embodiments, the process for preparing the solid, micellar compositions include the additional step of adding one or more additional lipid components to the solution. In some embodiments, the additional lipid components are added to the solution prior to the conversion of the cannabinoid acids to a salt form. In some embodiments, the additional lipid components are added to the solution prior to the emulsification of the cannabinoid acids to form the micelles. In some embodiments, the additional lipid components are added to the solution prior to filtering the micelles. In some embodiments, the additional lipid components are added to the solution prior to the addition of the metal to precipitate the micelles.

In some embodiments, the additional lipid components are cannabinoids and terpenes. In some embodiments, the additional lipid components are cannabinoids. In some embodiments, the additional lipid components are cannabinoids selected from the group consisting of THC, CBD, THCa, CBGa, CBG, CBN. In some embodiments, the additional lipid components are terpenes. In some embodiments, the additional lipid components are terpenes selected from the group consisting of α-pinene, myrcene, carophyllene oxide, limonene, linalool, β-carophyllene, α-humulene and terpinolene. In some embodiments, the additional lipid components are a mixture of cannabinoids and terpenes. In some embodiments, the additional lipid components are a mixture of cannabinoids and terpenes selected from the group consisting of THC, CBD, THCa, CBGa, CBG, CBN, α-pinene, myrcene, carophyllene oxide, limonene, linalool, β-carophyllene, α-humulene and terpinolene. In some embodiments, the additional lipid components are cannabinoids and/or terpenes, wherein the cannabinoids and terpenes are obtained from a plant concurrently with the cannabinoid acids that form the micelles. In some embodiments, the additional lipid components are cannabinoids and/or terpenes, wherein the cannabinoids and terpenes are added to the micelles.

In some embodiments, the additional lipid components are selected from the group consisting of CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, vitamin K. In some embodiments, the additional lipid components are selected from the group consisting of CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, vitamin K, and others.

In some embodiments, the step of converting the one or more cannabinoid acids to a salt form is performed by adding a base to the solution. In some embodiments, the base is sodium carbonate. In some embodiments, the step of converting the one or more cannabinoid acids to a salt form is performed by adding a base to the solution, wherein the base has a monovalent cation. In some embodiments, the monovalent cation is selected from the group consisting of lithium, sodium, potassium, rubidium, cesium, and ammonium. In some embodiments, the monovalent cation is lithium. In some embodiments, the monovalent cation is sodium. In some embodiments, the monovalent cation is potassium. In some embodiments, the monovalent cation is rubidium. In some embodiments, the monovalent cation is cesium. In some embodiments, the monovalent cation is ammonium.

In some embodiments, the step of emulsifying the salt form of the one or more cannabinoid acids to form the micelles is performed using high-shear mixing. In some embodiments, the resultant emulsion is stable and the desired components are stable as an emulsion for many days. In some embodiments, the resultant emulsion is stable and the desired components are stable as an emulsion for 3 days. In some embodiments, the resultant emulsion is stable and the desired components are stable as an emulsion for 7 days. In some embodiments, the resultant emulsion is stable and the desired components are stable as an emulsion for 14 days. In some embodiments, the resultant emulsion is stable and the desired components are stable as an emulsion for 21 days. In some embodiments, the resultant emulsion is stable and the desired components are stable as an emulsion for 30 days.

In some embodiments, the step of filtering the micelles separates undesired components in the crude extract from the CBDa salt micelles and incorporated lipids (including other cannabinoids). In some embodiments, the step of filtering the micelles is performed by filtration through Celite and subsequently rinsing the Celite with water.

In some embodiments, the metal added to precipitate the micelles is selected from the group consisting of an s-block metal, a d-block metal, and a p-block metal. In some embodiments, the metal is an s-block metal. In some embodiments, the metal is a d-block metal. In some embodiments, the metal is a p-block metal. In some embodiments, the metal added to precipitate the micelles is selected from the group consisting of magnesium, calcium, and strontium. In some embodiments, the metal is magnesium. In some embodiments, the metal is calcium. In some embodiments, the metal is strontium.

In some embodiments, the step of isolating the precipitate from the solution is performed via filtration. In some embodiments, the step of isolating the precipitate from the solution is performed via filtration through a frit. In some embodiments, the step of isolating the precipitate from the solution is performed via filtration through a frit, wherein the isolated precipitate is subsequently dried under reduced pressure.

### Methods of Using the Compositions of the Disclosure

Concurrent and synergistic delivery of cannabinoids and other active ingredients can be in the form of an oral, topical, ocular, or injectable application. By way of example, the anti-inflammatory properties of CBDa can work synergistically with the immunosuppressant, tacrolimus, for a more effective treatment of psoriasis. Thus, in some embodiments, topically applied lipophilic pharmaceuticals are incorporated into the CBDa salt micelle and retained in the chylobinoid, allowing a single application of the lipophilic drug and CBDa for a synergistic pharmacologic effect. In some embodiments, the topically applied lipophilic drugs are selected from the group consisting of tacrolimus, corticosteroids, rapamycin, cyclosporine, imiquimod, calcitriol, retinol, and others.

Marijuana-derived Epidiolex (100% CBD oil), the first antiepileptic drug (AED) approved by the FDA in many years, has shown efficacy in several forms of refractory epilepsy, but is known to have issues of poor and variable oral absorption requiring dosing ranges from 5 mg/kg/day to 50 mg/kg/day; potential adverse effects including fatigue, anorexia, transaminase elevation and insomnia; potential interactions with other medications through interactions with inhibitors or inducers of hepatic CYP3A4 or CYP2C19; and the possible development of tolerance in treated patients over time.

Chylobinoids (˜85% magnesium-CBDa) are a more efficacious and safer medication for the treatment of refractory epilepsy disorders for the following reasons.

Firstly, with respect to receptor affinity and effect, data from Parker et al has shown that CBDa itself is purportedly 1000-4000× as potent as CBD with a still undetermined additional potentiation by the chylobinoid micelle formulation described in this invention.

CBDa is more bioavailable than CBD (e.g. 2× according to Nahler) and chylobinoid may be 8-10× more bioavailable than CBD due to the enhanced amphiphilic properties of the metal-coordinated complex and avoidance of first-pass hepatic metabolism. Thus, in accordance with the principles disclosed herein, chylobinoids, which are designed to be more effectively absorbed from the gastrointestinal tract and selectively transported through the lymphatic system into the bloodstream, is significantly more bioavailable than CBD and CBDa.

Magnesium, a component of the chylobinoid micelle, is transported through the blood-brain barrier following therapeutic administration of the product, is a known NMDA receptor antagonist, and, as such, may potentiate the pharmacologic activity of CBDa at the NMDA receptor site and reduce NMDA-mediated excitability typically associated with neurodegenerative and neuroinflammatory disorders.

CBDa's intrinsic ability to selectively inhibit COX-2 receptors may further reduce the inflammation and excessive excitability associated with neurodegenerative disorders such as epilepsy, CNS infections, neurodegenerative disorders, and vascular diseases such as stroke.

Data has shown that CBDa demonstrates a 100-fold greater affinity for 5-HT_{1A} receptors over CBD which may also contribute to CBDa's increased anti-seizure activity as compared with CBD. Additionally, theoretical long-term anxiolytic and anti-depressant benefits of CBDa due to this increased 5-HT_{1A} affinity may improve cognition, mood, and the overall quality of life in these afflicted patients.

CDBa's known inhibitory effects on GPR-55, a recently discovered but poorly characterized cannabinoid receptor, may also have positive effects in epilepsy patients including improvement of their functional cognitive status. Antagonism of GPR-55 is associated with increased inhibitory neuron excitability.

The chylobinoid compositions disclosed herein can be formulated in a variety of ways. In some embodiments, the chylobinoid compositions disclosed herein can be formulated with an oil, such as coconut or sesame oil, into a cream or lotion for topical applications; into a spray for buccal absorption; or with water or saline for intravenous, intramuscular, intranasal, sublingual, or ophthalmic administration. Coconut oil, sesame oil or other food-based oils used to dissolve the chylobinoid products can be further put into capsules or tablets for oral administration.

In some embodiments, the chylobinoid compositions disclosed herein are formulated with coconut oil. In some embodiments, the chylobinoid compositions disclosed herein are formulated with sesame oil. In some embodiments, the chylobinoid compositions disclosed herein are formulated into a cream or lotion for topical applications. In some embodiments, the chylobinoid compositions disclosed herein are formulated into a spray for buccal absorption. In some embodiments, the chylobinoid compositions disclosed herein are formulated with water or saline for intravenous administration. In some embodiments, the chylobinoid compositions disclosed herein are formulated with water or saline for intramuscular administration. In some embodiments, the chylobinoid compositions disclosed herein are formulated with water or saline for intranasal administration. In some embodiments, the chylobinoid compositions disclosed herein are formulated with water or saline for sublingual administration. In some embodiments, the chylobinoid compositions disclosed herein are formulated with water or saline for ophthalmic administration.

In some embodiments, the chylobinoid compositions disclosed herein are formulated as pharmaceutically acceptable compositions comprising a chylobinoid composition and one or more pharmaceutically acceptable excipients. In some embodiments, the pharmaceutically acceptable excipient is Prosolv SMCC 90. In some embodiments, the pharmaceutically acceptable excipient is Explotab. In some embodiments, the pharmaceutically acceptable excipient is a mixture of Prosolv SMCC 90 and Explotab.

In some embodiments, the pharmaceutically acceptable composition is formulated as a powder comprising a chylobinoid composition and one or more pharmaceutically acceptable excipients. In some embodiments, the pharmaceutically acceptable excipient is Prosolv SMCC 90. In some embodiments, the pharmaceutically acceptable excipient is Explotab. In some embodiments, the pharmaceutically acceptable excipient is a mixture of Prosolv SMCC 90 and Explotab. In some embodiments, the powder formulation is prepared by blending the chylobinoid compositions and pharmaceutically acceptable excipients in a mortar and pestle.

In some embodiments, the chylobinoid compositions disclosed herein are formulated for oral administration. In some embodiments, the chylobinoid compositions disclosed herein are formulated for administration in an oral unit-dosage form. In some embodiments, the oral unit-dosage form is a capsule. In some embodiments, the capsule comprises the powder formulations disclosed herein. In some embodiments, the oral unit-dosage form is a tablet. In some embodiments, the oral unit-dosage form further comprises a food-based oil. In some embodiments, the food-based oil is coconut oil. In some embodiments, the food-based oil is sesame oil.

In some embodiments, the chylobinoid compositions disclosed herein are formulated for topical administration. In some embodiments, the chylobinoid compositions disclosed herein are formulated as a cream for topical administration. In some embodiments, the cream comprises a chylobinoid composition, MCT oil, and shea butter. In some embodiments, the cream comprises a chylobinoid composition, MCT oil, shea butter, and one or more essential oil fragrances. In some embodiments, the cream is prepared via processes comprising preparing a solution of the chylobinoid composition in MCT oil, melting shea butter to a creamy consistency and mixing with the chylobinoid/MCT oil solution to produce a mixture with a chylobinoid concentration of 1.5%, cooling the mixture to a near-solid state, warming the mixture to room temperature, mechanically mixing the mixture, and optionally adding essential oil fragrances and mechanically mixing the mixture.

Furthermore, the above described micelle formulation could be formed using THCa or CBGa instead of CBDa with magnesium chelation, and metal coordination complexes of THCa, CBGa and CBDa could be combined for additive or synergistic therapeutic purposes as well. Both THCa and CBDa have distinctive anti-inflammatory properties and receptor binding affinities, and as PPARy ligands their combination may be complementary and beneficial for the treatment of amyotrophic lateral sclerosis, Parkinson's disease, multiple sclerosis, stroke, and other neurodegenerative disorders. Other combinations of metal-coordinated CBDa, CBGa and THCa, along with CBD, THC, CBG, CBN and other natural cannabinoids, both with and without formulation as micelles, are also contemplated by this invention.

Methods of treating neurodegenerative disorders or pain comprising the step of administering to a patient in need thereof a therapeutically effective amount of a solid, micellar composition comprising micelles of one or more cannabinoid acids and a metal, wherein the one or more cannabinoid acids are in a salt form, the salt form having a monovalent counter ion, and the micelles being free of added surfactants, are disclosed herein.

In some embodiments, the neurodegenerative disorder to be treated is selected from the group consisting of amyotrophic lateral sclerosis, Parkinson's disease, multiple sclerosis, and stroke. In some embodiments, the neurodegenerative disorder is amyotrophic lateral sclerosis. In some embodiments, the neurodegenerative disorder is Parkinson's disease. In some embodiments, the neurodegenerative disorder is multiple sclerosis. In some embodiments, the neurodegenerative disorder is stroke.

In some embodiments, the pain to be treated is selected from neuropathic pain and arthritic pain. In some embodiments, the pain to be treated is neuropathic pain. In some embodiments, the pain is arthritic pain.

In some embodiments, the one or more cannabinoid acids selected from the group consisting of cannabidolic acid (CBDa), tetrahydrocannabinolic acid (THCa) and cannabigerolic acid (CBGa). In some embodiments, the cannabinoid acid is CBDa. In some embodiments, the cannabinoid acid is THCa. In some embodiments, the cannabinoid acid is CBGa. In some embodiments the solid micellar compositions comprise micelles of a combination of CBDa, THCa, and/or CBGa.

In some embodiments, the monovalent counter ion of the salt form of the cannabinoid acid is selected from the group consisting of lithium, sodium, potassium, rubidium, cesium, and ammonium. In some embodiments, the monovalent counter ion is lithium. In some embodiments, the monovalent counter ion is sodium. In some embodiments, the monovalent counter ion is potassium. In some embodiments, the monovalent counter ion is rubidium. In some embodiments, the monovalent counter ion is cesium. In some embodiments, the monovalent counter ion is ammonium.

In some embodiments, the metal is selected from an s-block metal, a d-block metal, and a p-block metal. In some embodiments, the metal is an s-block metal. In some embodiments, the metal is a d-block metal. In some embodiments, the metal is a p-block metal. In some embodiments, the metal is selected from the group consisting of magnesium, calcium, and strontium. In some embodiments, the metal is magnesium. In some embodiments, the metal is calcium. In some embodiments, the metal is strontium.

In some embodiments, the solid, micellar compositions further comprise one or more additional lipid components, wherein the additional lipid components are sequestered within the hydrophobic core of the micelles.

In some embodiments, the additional lipid components are cannabinoids and terpenes. In some embodiments, the additional lipid components are cannabinoids. In some embodiments, the additional lipid components are cannabinoids selected from the group consisting of THC, CBD, THCa, CBGa, CBG, CBN. In some embodiments, the additional lipid components are terpenes. In some embodiments, the additional lipid components are terpenes selected from the group consisting of α-pinene, myrcene, carophyllene oxide, limonene, linalool, β-carophyllene, α-humulene and terpinolene. In some embodiments, the additional lipid components are a mixture of cannabinoids and terpenes. In some embodiments, the additional lipid components are a mixture of cannabinoids and terpenes selected from the group consisting of THC, CBD, THCa, CBGa, CBG, CBN, α-pinene, myrcene, carophyllene oxide, limonene, linalool, β-carophyllene, α-humulene and terpinolene. In some embodiments, the additional lipid components are cannabinoids and/or terpenes, wherein the cannabinoids and terpenes are obtained from a plant concurrently with the cannabinoid acids that form the micelles. In some embodiments, the additional lipid components are cannabinoids and/or terpenes, wherein the cannabinoids and terpenes are added to the micelles.

In some embodiments, the additional lipid components are selected from the group consisting of CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, vitamin K. In some embodiments, the additional lipid components are selected from the group consisting of CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, vitamin K, and others.

The present disclosure enables one of skill in the relevant art to make and use the inventions provided herein in accordance with multiple and varied embodiments. Various alterations, modifications, and improvements of the present disclosure that readily occur to those skilled in the art, including certain alterations, modifications, substitutions, and improvements are also part of this disclosure. Accordingly, the foregoing description are by way of example to illustrate the discoveries provided herein.

### EXAMPLES

### Example 1—Hemp Extraction with Acetone

In a mixing bowl, 700 mL of acetone was added to 500 g of dry ice, and 250 g of hemp was subsequently added. The mixture was allowed to steep for approximately five minutes with periodic stirring. The extracts were passed through a 20 cm ceramic filter funnel lined with 10 μm filter paper that was fitted to a 3 L side-arm flask under vacuum. The hemp flower captured in the filter funnel was rinsed with two washes of chilled acetone (200 mL). The acetone was removed in vacuo and under high vacuum until the gurgling stopped to yield a golden brown, thick hemp oil syrup.

### Example 2—Conversion of Hemp Oil to Chilobinoid

Hemp oil (˜70% CBDa, 18.5 g of oil, 12.9 g CBDa, 36.1 mmol of CBDa) was added to a 3 L round bottom flask. Hot tap water (˜50° C., 1500 mL) was added along with sodium carbonate (7.59 g, 72.2 mmol). The mixture was homogenized for 5 minutes on high and then placed in the refrigerator for 1 hour to cool. The mixture was filtered through a frit packed with Celite. A solution of magnesium chloride (15.89 g, 72.2 mmol, 100 mL) was added to the filtrate. Pinkish white solid formed, which was filtered through a frit, and dried under reduced pressure to yield 11.6 g pale purple solid (87%).

Analysis: Detected cannabinoids: 71.8% CBDa, 5.1% CBD, 5.1% CBGa, 1.9% THCa, 0.2% CBC

### Example 3—Preparation of Curcumin Infused Chylobinoid

Hemp oil (˜64% CBDa, 650 mg of oil, 377 mg CBDa, 1.05 mmol of CBDa) was added to a tall jar. Hot tap water (˜50° C., 70 mL) was added, along with sodium carbonate (332 mg, 3.06 mmol) and curcumin (387 mg, 3.06 mmol). The mixture was homogenized for 5 minutes on high and then placed in the refrigerator for 2 hrs to cool. The mixture was filtered through cheese cloth. A solution of magnesium chloride (673.2 mg, 2.1 mmol, 3 mL) was added to the filtrate dropwise. An orange solid formed and was filtered through a frit and dried under reduced pressure to yield 739 mg orange solid.

Analysis: 27% CBDa

### Example 4—Preparation of Large Scale Chylobinoid

Hemp oil (˜65% CBDa, 286 g of oil, 185.9 g CBDa, 0.519 mol of CBDa) was added to a 20 L rectangular bottom stainless steel pan. Hot tap water (˜50° C., 13 L) was added, along with sodium carbonate (110 g, 1.036 mol). The mixture was homogenized for 5 minutes on high and then cooled with 64 ounce rapid cool wands. The mixture was filtered through a Buchner funnel packed with Celite. A solution of magnesium chloride (228.48 g, 1.038 mmol, 400 mL) was added to the filtrate. An off-white solid formed, which was filtered through a Buchner funnel and dried under reduced pressure to yield a light tan powder (185.79 g).

### Example 5—Powder Formulations of Chylobinoids and Pharmaceutically Acceptable Excipients

A powder formulation of chylobinoids and a pharmaceutically acceptable excipient were prepared for the manufacture of capsules containing 15 mg of chylobinoid.

The powder formulation was prepared using the following components:

| | | |
|---|---|---|
| Chylobinoid | 59 | g |
| Prosolv SMCC 90 | 225.29 | g |
| Explotab | 16.96 | g |

To a mortar, approximately 20 g of Prosolv was added, followed by approximately 5 g of Chylobinoid and 2 g of Explotab. The Prosolv is added first to prevent the Chylobinoid from sticking. The mixture was ground with the pestle thoroughly until no streaks of powder were observed. Additional Prosolv, Chylobinoid, and Explotab were added in the same proportions identified above and ground with pestle, allowing a 5-10 minute interval between each addition. The sides and bottom of the mortar were periodically scraped with a spatula to ensure that no powder was sticking to the mortar. Samples were subsequently analyzed to ensure that the powder formulation was uniform before being packaged in capsules.

Analysis: Detected Cannabinoids: 10.76% CBDa, 0.83% CBD, 0.32% THCa

### Example 6—Cream Formulations of Chylobinoids

Chylobinoid powder was mixed with MCT oil and the resulting solution was analyzed to determine the CBDa content of the solution. The solution was subsequently used to prepare a cream formulation containing a chylobinoid concentration of 1.5% via the following protocol.

Shea butter was melted to a creamy consistency and mixed with the chylobinoid and MCT oil solution in a beaker to produce a mixture with a chylobinoid concentration of 1.5%. The mixture was then chilled in a refrigerator until the mixture was in a nearly solid form. The near-solid mixture was subsequently warmed to room temperature, added to the bowl of a stand mixer, and mixed for 45 minutes. Essential oil fragrances were subsequently added and the mixture was mixed for an additional 15-30 minutes at medium-low speed to produce a cream with a light and fluffy texture. The cream was subsequently piped via pastry bag into 1 and 2 ounce jars and sealed.

1. A solid, micellar composition, comprising micelles of one or more cannabinoid acids and a metal, wherein:
the one or more cannabinoid acids are in a salt form, the salt form having a monovalent counter ion;
the micelles are free of added surfactants; and
the micelles comprise an outer hydrophilic portion and an inner hydrophobic portion.

2. The composition of claim 1, wherein the one or more cannabinoid acids are selected from the group consisting of: CBDa, THCa, and CBGa.

3. The composition of claim 1, wherein the monovalent cation is selected from the group consisting of: lithium, sodium, potassium, rubidium, cesium, and ammonium.

4. The composition of claim 1, wherein the metal is selected from the group consisting of: an s-block metal, a d-block metal, and a p-block metal.

5. The composition of claim 4, wherein the metal is selected from the group consisting of: magnesium, calcium, and strontium.

6. The composition of claim 1, further comprising one or more additional lipid components, wherein the additional lipid components are sequestered within the inner hydrophobic portion of the micelles.

7. The composition of claim 6, wherein the one or more additional lipid components are selected from cannabinoids and terpenes, or a combination thereof, wherein the cannabinoids and terpenes are obtained from a plant concurrently with the one or more cannabinoid acids.

8. The composition of claim 7, wherein the cannabinoids and terpenes are selected from the group consisting of: THC, CBD, THCa, CBGa, CBG, CBN, α-pinene, myrcene, carophyllene oxide, limonene, linalool, β-carophyllene, α-humulene and terpinolene.

9. The composition of claim 6, wherein the one or more additional lipid components are selected from the group consisting of: CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, and vitamin K.

10. A process for preparing a solid, micellar composition comprising micelles of one or more cannabinoid acids and a metal, wherein the one or more cannabinoid acids are in a salt form, the salt form having a monovalent counter ion, and the micelles being free of added surfactants, the process comprising:
adding the one or more cannabinoid acids to a solution comprising water;
converting the one or more cannabinoid acids to a salt form;
emulsifying the salt form of the one or more cannabinoid acids to form the micelles;
filtering the micelles;
adding the metal to the micelles to form a precipitate; and
isolating the precipitate from the solution.

11. The process of claim 10, wherein the one or more cannabinoid acids are selected from the group consisting of CBDa, THCa, and CBGa.

12. The process of claim 10, wherein the micellar composition further comprises one or more additional lipid components selected from cannabinoids and terpenes, or a combination thereof, wherein the cannabinoids and terpenes are obtained from a plant concurrently with the one or more cannabinoid acids.

13. The process of claim 12, wherein the cannabinoids and terpenes are selected from the group consisting of: THC, CBD, THCa, CBGa, CBG, CBN, α-pinene, myrcene, carophyllene oxide, limonene, linalool, β-carophyllene, α-humulene and terpinolene.

14. The process of claim 10, further comprising adding one or more additional lipid components to the solution, wherein the one or more additional lipid components are selected from the group consisting of: CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, and vitamin K.

15. The process of claim 10, wherein converting the one or more cannabinoid acids to a salt form comprises adding a base to the solution, the base having a monovalent cation.

16. The process of claim 15, wherein the monovalent cation is selected from the group consisting of: lithium, sodium, potassium, rubidium, cesium, and ammonium.

17. The process of claim 10, wherein emulsifying the salt form of the one more cannabinoid acids to form micelles comprises high-shear mixing.

18. The process of claim 10, wherein the metal is selected from the group consisting of: an s-block metal, a d-block metal, and a p-block metal.

19. The process of claim 18, wherein the metal is selected from the group consisting of: magnesium, calcium, and strontium.

20. A method of treating a neurodegenerative disorder or pain, wherein the neurodegenerative disorder is selected from amyotrophic lateral sclerosis, Parkinson's disease, multiple sclerosis, and stroke, and the pain is selected from neuropathic pain and arthritic pain, comprising the step of administering to a patient in need thereof a therapeutically effective amount of a micellar composition comprising micelles of one or more cannabinoid acids and a metal, wherein the one or more cannabinoid acids are in a salt form, the salt form having a monovalent counter ion, and the micelles being free of added surfactants.

21. The method of claim 20, wherein the one or more cannabinoid acids are selected from the group consisting of: CBDa, THCa, and CBGa.

22. The method of claim 20, wherein the metal is selected from the group consisting of: magnesium, calcium, and strontium.

23. The method of claim 20, wherein the micellar composition further comprises one or more additional lipid components selected from the group consisting of: THC, CBD, THCa, CBGa, CBG, CBN, α-pinene, myrcene, carophyllene oxide, limonene, linalool, β-carophyllene, α-humulene, terpinolene, CoQ10, omega-3, omega-6, nicotine, resveratrol, tocotrienols, flavonoids, gamma-tocopherols, steroids, lipophilic antibiotics, tacrolimus, curcumin, vitamin A, vitamin B, vitamin D, and vitamin K, wherein the additional lipid components are sequestered within an inner hydrophobic portion of the micelles.
